(19) United States
(12) Patent Application Publication
(10) Pub. No.: US 2016/0331912 A1
(43) Pub. Date: Nov. 17, 2016

(54) Device for Vaporization of Phyto Material

(71) Applicant: MICHAEL ALEXANDER TRZECIESKI, MID LEVELS (HK)

(72) Inventor: MICHAEL ALEXANDER TRZECIESKI, MID LEVELS (HK)

(21) Appl. No.: 15/140,730

(22) Filed: Apr. 28, 2016

Related U.S. Application Data
(60) Provisional application No. 62/161,079, filed on May 13, 2015.

(51) Int. Cl. A61M 11/04

(52) U.S. Cl. CPC A61M 11/042; A61M 2205/582; A61M 2205/3368; A61M 2205/50; A61M 2205/581

(57) ABSTRACT

A novel device for vaporization is disclosed that includes vibration notification as well as an airflow processing member and a heating chamber that is not proximate an inhalation aperture from which the user inhales the vapor. This allows for less hot vapors to be inhaled by the end user and provides for a vaporization device that is more useable by those with macular degeneration.

### CROSS REFERENCE TO RELATED APPLICATION

This application benefits from the priority of U.S. Provisional Application 62/161,079 filed on May 13, 2015, which is incorporated herein by reference.

### TECHNICAL FIELD OF THE INVENTION

The technical field relates to a device that provides for vaporization of phyto materials.

### BACKGROUND OF THE INVENTION

There are a large number of vision related diseases, which range from extremely common, Macular Degeneration and Glaucoma, to rare ones, such as Muscular Dystrophy and Stargardt's disease, to which suffers experience symptoms such as loss of central vision, blurred vision, especially while reading, distorted vision and colors that look faded or are indistinguishable. Of course other diseases may lead to loss of hearing in addition to the possibility of blindness.

Macular Degeneration is the most common cause of blindness in people over age 60. However, many are devastating conditions that often affect people in the prime of life. There are a large number of adaptive devices that can help people see, these include magnifying glasses, special eyeglass lenses, computer screen readers and TV systems that enlarge reading material. There is a recent boom in large screen smartphones, where these larger screens are used to facilitate use by those that are experiencing vision related diseases. In some cases where the macular degeneration is bad users must rely on their sense of touch in order to operate simple devices around them. There are various ailments, such as the ones mentioned and others for which aromatherapy is prescribed as a treatment option by physicians.

Aromatherapy generally uses essential oils, which are extracted from phyto materials, such as leaves of plants, for therapeutic benefits. These essential oils are either massaged into the skin or can be inhaled. In some cases the phyto materials are heated in order to released the essential oils therefrom. By heating these phyto materials at predetermined temperatures, essential oils and extracts are boiled off, depending upon the temperature at which these phyto materials are heated, a vapor, which is a faintly visible suspension of fine particles of matter in the air or aerosol, which is a gaseous suspension of fine solid or liquid particles, is given off, which is then inhaled by a user for its therapeutic benefits.

Devices that provide such operation are generally known as vaporizers and they generally fall into two categories. These are convection and conduction. Convection vaporizers pass hot air at a predetermined temperature through the ground leaf materials to extract the various essential oils to generate the vapor, whereas conduction vaporizers provide heat to the phyto materials through direct contact between the phyto materials and a heating chamber to generate the vapor.

Different phyto materials release vapor at different temperatures. Some release vapor at 120 degrees Celsius, whereas others at 220 degrees Celsius. The predetermined temperature is less than a combustion temperature of the phyto material. In some of the prior art vaporization devices the vapor released from the materials are quite hot, around 230 degrees Celsius and may cause discomfort when inhaled by a user, which is typically a result of the close proximity of the users lips to heating source used for vaporizing of the phyto material. In many prior art vaporizer devices, the heating chamber is very close to the lips of the user, in some cases less than 2 centimeters away. This means that the hot air and vapor mixture may easily cause discomfort as well as potentially to burn the lips of the user. Furthermore, some users who suffer from macular degeneration may not have sufficient vision to be able to interact with their vaporizer for administering of the aromatherapy, hence using other than visual cues proves to be advantageous. In addition, many prior art vaporization devices allow for no restricted airflow and the user can inhale a lot of air and not a lot of vapors that are released from the phyto material. The inhalation of a lot of hot air as well as the hot air not having much phyto material vapors contained therein is not advantageous.

It is therefore an object of the invention to provide a phyto material vaporization device that overcomes the deficiencies in the prior art.

### SUMMARY OF THE INVENTION

In accordance with the invention there is provided a device for vaporizing of phyto material and adapted to fit into a pocket comprising: a housing comprising a first end and a second end opposite the first end; a heating chamber for receiving of phyto material disposed upstream and at the first end and an inhalation aperture proximate a second end thereof, the heating chamber comprising a first aperture and comprising a second aperture disposed at an opposite end thereof and downstream from the first aperture, and comprising a heating chamber fluid pathway formed between the first aperture and the second aperture; a first fluid pathway for receiving of ambient air disposed upstream of the first aperture; a second fluid pathway fluidly coupled with the inhalation aperture and downstream of the second aperture; an airflow processing member for restricting a flow of ambient air through the first fluid pathway and being releasably coupled at the first end proximate the heating chamber for processing of ambient air that flows through the heating chamber, wherein when the airflow processing member is coupled at the first end proximate the heating chamber comprising a continuous fluid pathway formed from the first fluid pathway through the heating chamber fluid pathway and into the second fluid pathway and when the airflow processing member is other than coupled at the first end proximate the heating chamber other than comprising the continuous fluid pathway for allowing loading and unloading of the phyto material into the heating chamber; at least one of a convection heating element and a conduction heating element disposed upstream of the inhalation aperture for heating the phyto material to release a vapor from the phyto material for flowing through the second fluid pathway for inhalation through the inhalation aperture; a temperature sensor thermally coupled proximate the heating chamber for providing a temperature control signal; a first rechargeable battery; a first control circuit comprising a processor, wherein the first control circuit is electrically coupled with the first rechargeable battery and electrically coupled with the at least one of a convection heating element and a conduction heating element, the first control circuit for controlling a flow of electrical current from the first rechargeable battery to the at least a heating element in dependence upon the a temperature control signal and for measuring a voltage level from the first rechargeable battery and for providing a battery voltage level signal; a switch for receiving of tactile input and electrically coupled with the first control circuit for providing a switch control signal to the first control circuit; a charging port electrically coupled with the first control circuit, the charging port for receiving of electrical energy and for controllably providing of the received electrical energy to the first rechargeable battery; and, a vibration notification system electrically coupled with the first control circuit and mechanically coupled with one of the housing and the airflow processing member for transmitting of vibration thereto, the vibration notification system for providing the vibration in dependence upon at least one of the switch control signal and the battery voltage level signal and the temperature control signal, wherein the vibration is for at least partially vibrating of the one of the housing and the airflow processing member.

In accordance with the invention there is provided a device for vaporizing of phyto material and adapted to fit into a pocket comprising: a housing comprising a first end and a second end opposite the first end; a heating chamber for receiving of a phyto material disposed at the first end and an inhalation aperture proximate a second end thereof, the heating chamber comprising a first aperture and a second aperture disposed at an opposite end thereof and downstream from the first aperture, a heating chamber fluid pathway formed between the first aperture and the second aperture; a first fluid pathway for receiving of ambient air disposed upstream of the first aperture; a second fluid pathway fluidly coupled with the inhalation aperture and downstream of the second aperture; an airflow processing member for being releasably coupled at the first end proximate the heating chamber for processing of ambient air that flows through the heating chamber, wherein when the airflow processing member is coupled at the first end proximate the heating chamber a continuous fluid pathway is formed from the first fluid pathway through the heating chamber fluid pathway and into the second fluid pathway and when the airflow processing member is other than coupled at the first end proximate the heating chamber an other than continuous fluid pathway is formed and allows for loading and unloading of the phyto material into the heating chamber; a conduction heating element disposed upstream of the inhalation aperture and thermally coupled with the heating chamber for conduction heating of the phyto material to release a vapor therefrom for flowing through the second fluid pathway for inhalation through the inhalation aperture, wherein coupling of the airflow processing member at the first end proximate the heating chamber for processing of ambient air that flows through the heating chamber comprises restricting a flow of ambient air through the first fluid pathway into the heating chamber; a temperature sensor thermally coupled proximate the heating chamber for providing a temperature control signal; a first rechargeable battery; a first control circuit comprising a processor, wherein the first control circuit is electrically coupled with the first rechargeable battery and electrically coupled with the heating element, the first control circuit for controlling a flow of electrical current from the first rechargeable battery to the heating element and for measuring a voltage level from the first rechargeable battery and for providing a battery voltage level signal; a switch for receiving of tactile input and electrically coupled with the first control circuit for providing a switch control signal to the first control circuit; a charging port electrically coupled with the first control circuit, the charging port for receiving of electrical energy and for controllably providing of the received electrical energy to the first rechargeable battery; and, a vibration notification system electrically coupled with the first control circuit and mechanically coupled with one of the housing and the airflow processing member for transmitting of vibration thereto, the vibration notification system for providing the vibration in dependence upon at least one of the switch control signal and the battery voltage level signal and the temperature control signal, wherein the vibration is for at least partially vibrating of the one of the housing and the airflow processing member.

In accordance with the invention there is provided A device for vaporizing of phyto material and adapted to fit into a pocket comprising: a housing comprising a first end and a second end opposite the first end; a heating chamber for receiving of a phyto material disposed at the first end and an inhalation aperture proximate a second end thereof, the heating chamber comprising a first aperture and a second aperture disposed at an opposite end thereof, a heating chamber fluid pathway formed between the first aperture and the second aperture; a first fluid pathway for receiving of ambient air disposed upstream of the first aperture; a second fluid pathway fluidly coupled with the inhalation aperture and downstream of the second aperture; an airflow processing member for being releasably coupled at the first end proximate the heating chamber for processing of ambient air that flows through the heating chamber, wherein when the airflow processing member is coupled at the first end proximate the heating chamber a continuous fluid pathway is formed from the first fluid pathway through the heating chamber fluid pathway and into the second fluid pathway and when the airflow processing member is other than coupled at the first end proximate the heating chamber an other than continuous fluid pathway is formed and allows for loading and unloading of the phyto material into the heating chamber; a convection heating element thermally coupled with the first fluid pathway and disposed upstream of the inhalation aperture for processing of ambient air by convection heating air flowing through the first fluid pathway for providing of heated air into the heating chamber for contacting the phyto material to release a vapor from the phyto material for flowing through the second fluid pathway for inhalation through the inhalation aperture when the airflow processing member is coupled at the first end proximate the heating chamber; a temperature sensor thermally coupled proximate the heating chamber for providing a temperature control signal; a first rechargeable battery; a first control circuit comprising a processor, wherein the first control circuit is electrically coupled with the first rechargeable battery and electrically coupled with the heating element, the first control circuit for controlling a flow of electrical current from the first rechargeable battery to the heating element and for measuring a voltage level from the first rechargeable battery and for providing a battery voltage level signal; a switch for receiving of tactile input and electrically coupled with the first control circuit for providing a switch control signal to the first control circuit; a charging port electrically coupled with the first control circuit, the charging port for receiving of electrical energy and for controllably providing of the received electrical energy to the first rechargeable battery; and, a vibration notification system electrically coupled with the first control circuit and mechanically coupled with one of the housing and the airflow processing member for transmitting of vibration thereto, the vibration notification system for providing the vibration in dependence upon at least one of the switch control signal and the battery voltage level signal and the temperature control signal, wherein the vibration is for at least partially vibrating of the one of the housing and the airflow processing member.

### BRIEF DESCRIPTION OF THE DRAWINGS

Exemplary embodiments of the invention will now be described in conjunction with the following drawings, in which:

FIG. 1A illustrates a perspective view of a device for vaporization (DFV) in accordance with a first embodiment of the invention;

FIG. 1B illustrate a side view of a DFV in accordance with a first embodiment of the invention;

FIG. 1C illustrates a releasable coupling of an airflow processing member to the housing using magnets;

FIG. 1D illustrates a charging port as part of the DFV

FIG. 1E illustrates an exploded view of the DFV;

FIG. 1F illustrates a processor disposed as part of the first control circuit and comprising a Bluetooth® module;

FIG. 1G illustrates the second fluid pathway having an angled bend along its length;

FIG. 1H, illustrates the second fluid pathway having an angled bend along its length and with thermal insulation material;

FIG. 1I illustrates a conduction heating element disposed about the heating chamber and a vibration notification system;

FIG. 1J illustrates an audio microphone mechanically coupled with the second fluid pathway;

FIG. 1K illustrates a tethered connection between the airflow processing member in accordance with a second embodiment of the invention;

FIG. 1L illustrates an electrical connection for electrically coupling of the airflow processing member to the first control circuit 110 and to a housing of the DFV;

FIG. 2A illustrates a two part second fluid pathway in accordance with a third embodiment of the invention;

FIG. 2B illustrates separating of the two part second fluid pathway for allowing for cleaning thereof;

FIG. 2C illustrates cooling fins for increases a surface area of a second portion for contacting ambient air;

FIG. 3A illustrates a convection heating element disposed within the airflow processing member as part of a fourth embodiment of the invention.

FIG. 3B illustrates an exploded view of the airflow processing member having the conduction heating element disposed therein;

FIG. 3C illustrates another view of the conduction heating element;

FIG. 3D illustrates the DFV in accordance with the fourth embodiment of the invention;

FIG. 4A illustrates the DFV in accordance with a fifth embodiment of the invention; and

FIG. 4B illustrates an other than continuous fluid pathway being formed and allows for loading and unloading of phyto material 419 into the heating chamber.

### DETAILED DESCRIPTION OF EMBODIMENTS OF THE INVENTION

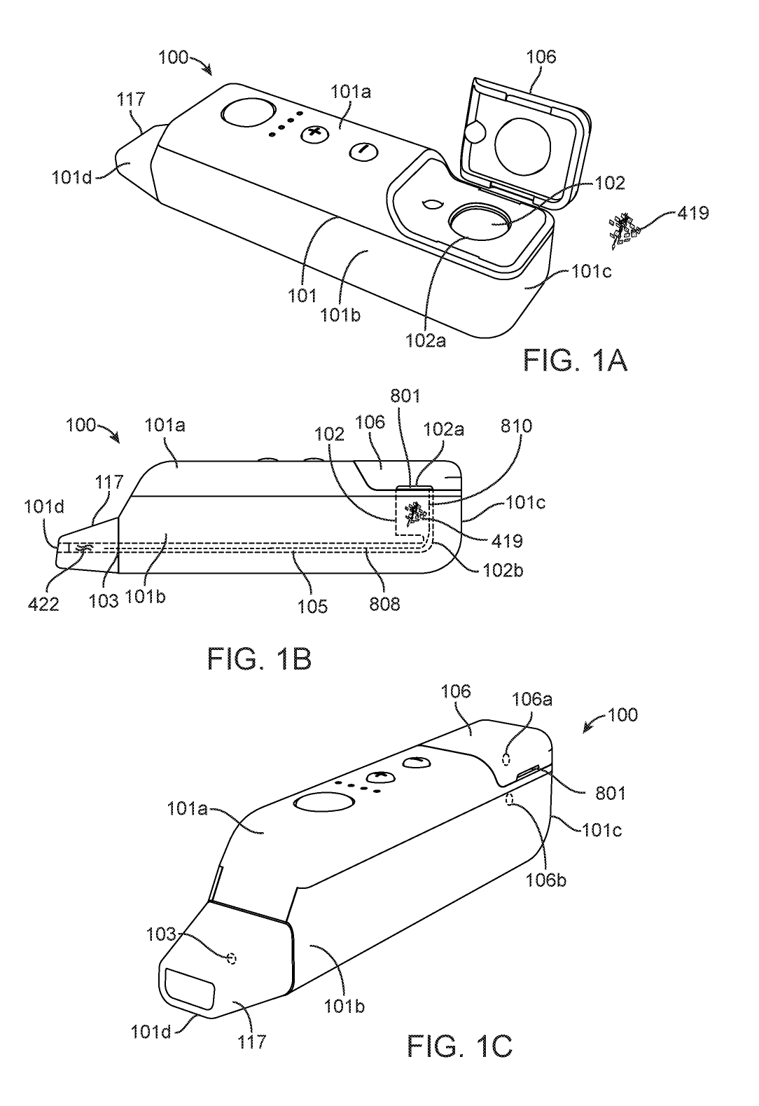

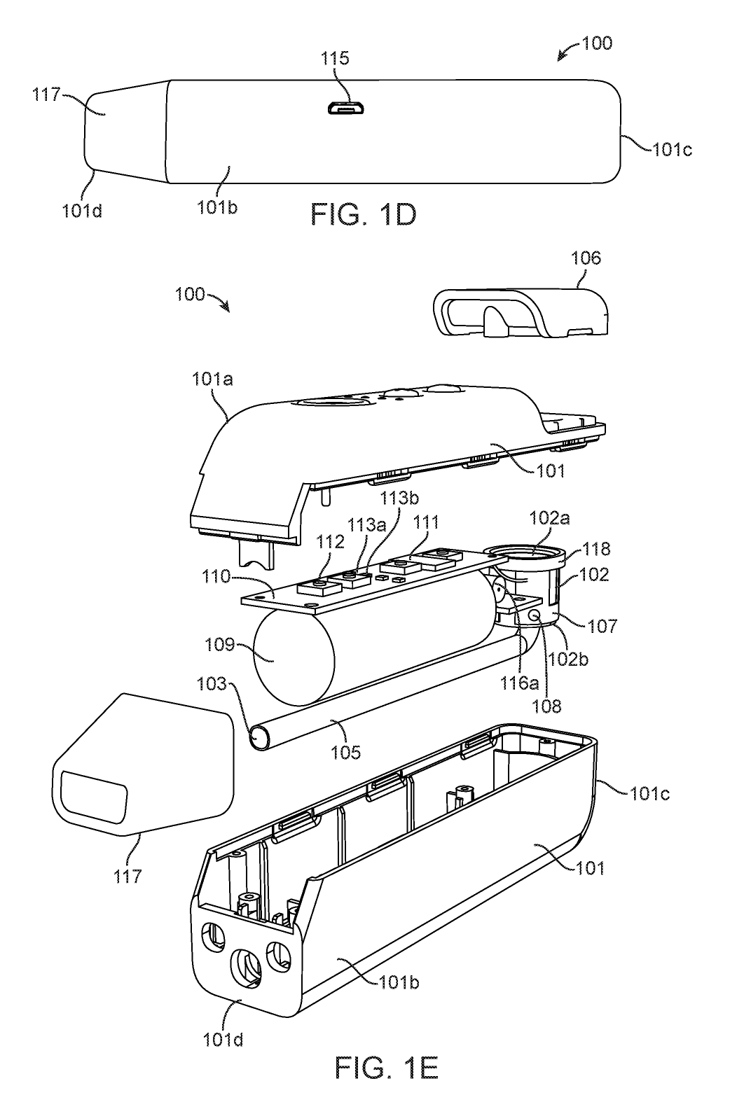

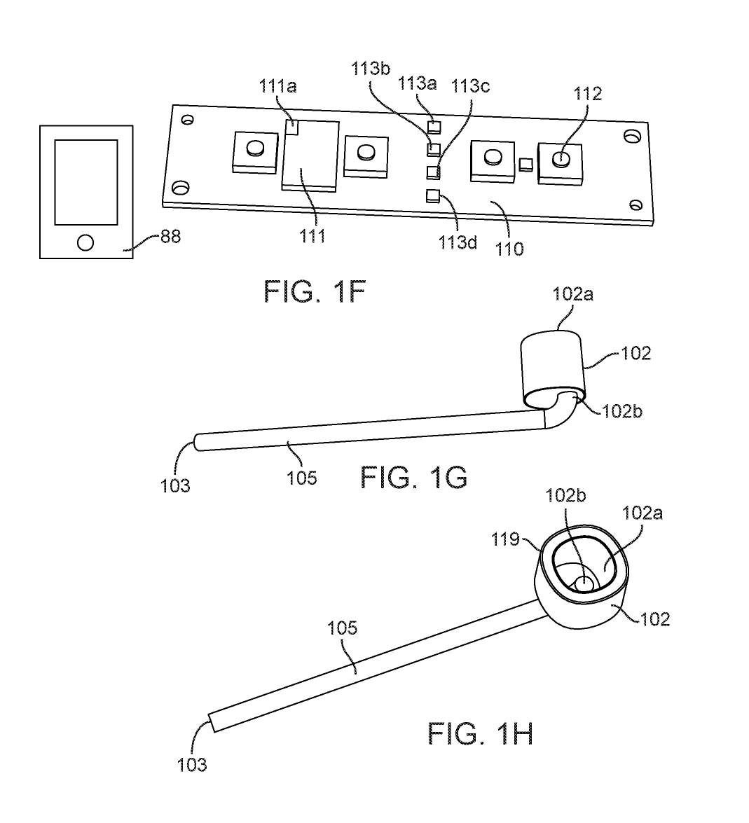

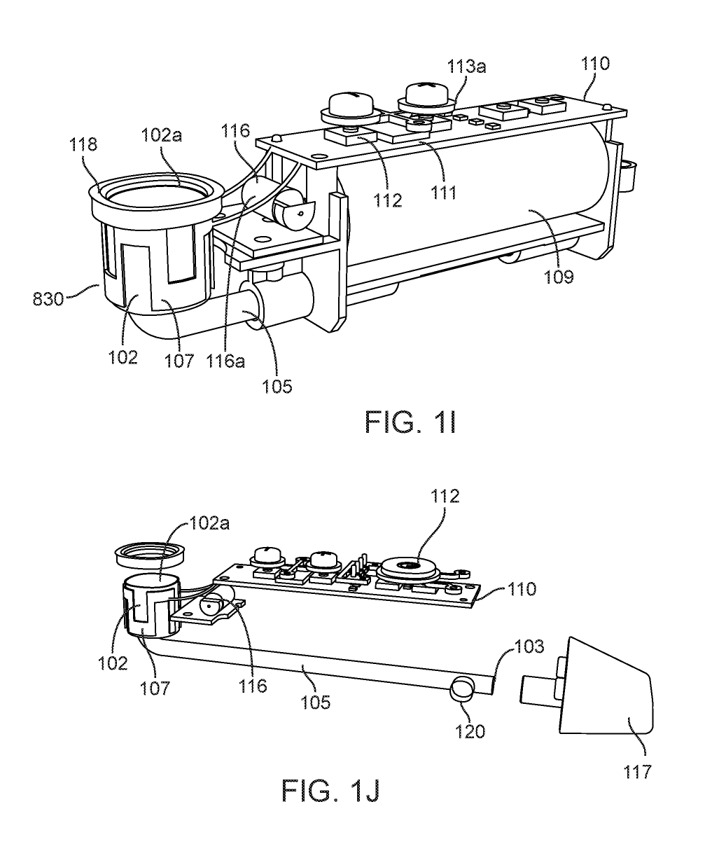

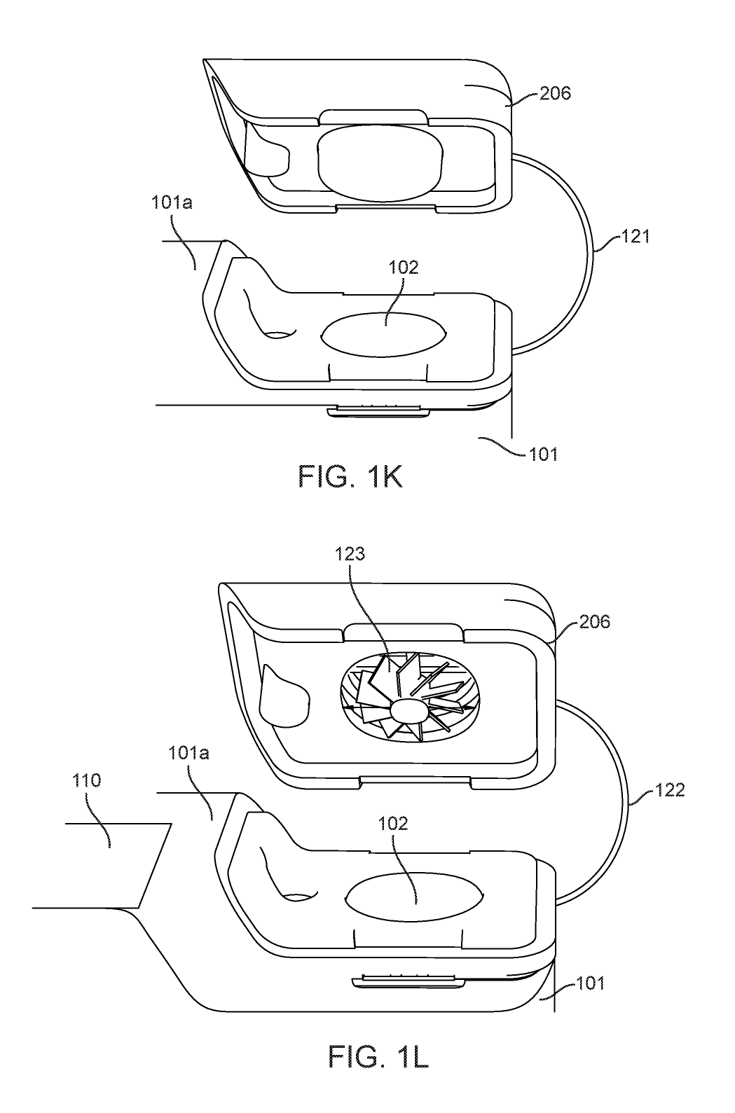

FIGS. 1A and 1B illustrate a perspective view and a side view, respectively, of a device for vaporization (DFV) 100 of phyto material and adapted to fit into a pocket in accordance with a first embodiment of the invention. The DFV 100 is formed from a housing 101 comprising a first end 101c and a second end 101d opposite the first end 101c. A heating chamber 102 for receiving of phyto material 419 is disposed at the first end 101c and an inhalation aperture 103 is disposed proximate a second end 101d thereof, the heating chamber 102 being upstream of the inhalation aperture 103. The heating chamber 102 is for heating of the phyto material 419 for releasing a vapor 422 therefrom for inhalation from the inhalation aperture 103.

The heating chamber 102 comprising a first aperture 102a upstream of a second aperture 102b (FIG. 1B) disposed at an opposite end thereof. A heating chamber fluid pathway 810 formed between the first aperture 102a and the second aperture 102b. A first fluid pathway 801 for receiving of ambient air is provided upstream of a second fluid pathway 105 is fluidly coupled with the inhalation aperture 103. Preferably the separation between the first end 101c and the second end 101d is at least 4 cm in order for a user not to get too much heat from having their lips too close to the heating chamber 102. Having such a distance allows for cooling of the vapor 422 along the second fluid pathway 105.

An airflow processing member 106 is provided for being releasably coupled at the first end 101c proximate the heating chamber 102 for processing of ambient air that flows through the heating chamber 102. When the airflow processing member 106 is coupled at the first end 101c proximate the heating chamber (FIG. 1B) a continuous fluid pathway 808 is formed from the first fluid pathway 801 through the heating chamber fluid pathway 810 and into the second fluid pathway 105 and a flow of ambient air that flows through the first fluid pathway 801 is restricted. When the airflow processing member 106 is other than coupled (FIG. 1A) at the first end 101c proximate the heating chamber 102 an other than continuous fluid pathway is formed and allows for loading and unloading of the phyto material 419 into the heating chamber 102. Phyto material 419 is inserted into the heating chamber 102 between the first and second apertures, 102a and 102b, as is shown in FIG. 1B.

In the case of FIG. 1A, the airflow processing member 106 is shown being uncoupled with the heating chamber first aperture 102a and referring to FIG. 1B, the airflow processing member 106 is shown being coupled with the heating chamber first aperture 102a. Referring to FIG. 1B the second fluid pathway 105 is shown in dashed lines as disposed within the housing 101. When the airflow processing member 106 is coupled with the heating chamber first aperture 102a, a small gap is formed between the airflow processing member 106 and the heating chamber first aperture 102a, allowing for restricted airflow into the heating chamber 102 when suction is applied to the inhalation aperture 103 by the user of the DFV 100. Preferably the heating chamber 102 is in the form of bowl for surrounding of the phyto material 419.

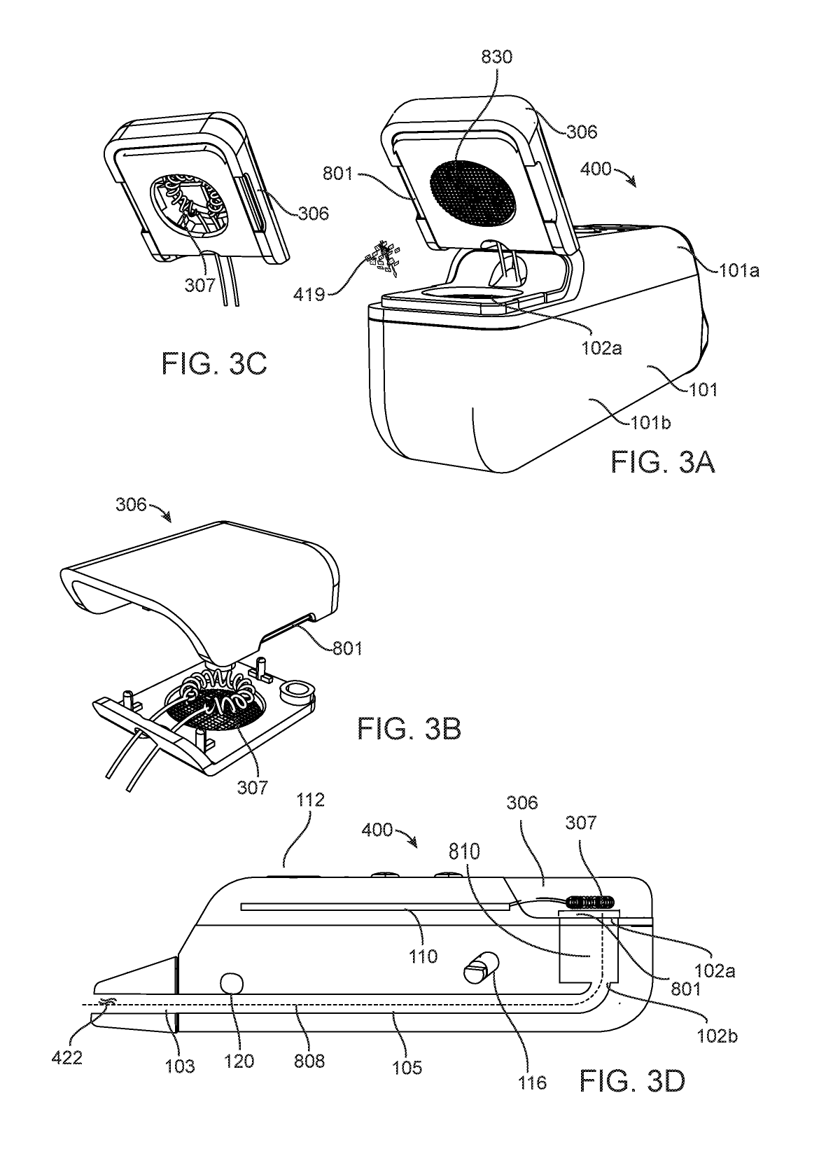

Referring to FIGS. 1I and 3A, at least a heating element 830 is provided for heating of the phyto material 419 through at least one of a convection heating (FIG. 3A) and conduction heating (FIG. 1I) to release a vapor from the phyto material 419 for flowing through the second fluid pathway 105 for inhalation through the inhalation aperture 103 by a user. Preferably the heating by convection or conduction is between 160 degrees Celsius and 230 degrees Celsius.

FIG. 1J illustrates a conduction heating element 107 thermally coupled with the heating chamber 102 for conduction heating of the phyto material 419 through transfer of internal energy by a microscopic collision of particles from the conduction heating element 107 through the heating chamber 102 and into the phyto material 419. The conduction heating element 107 contacts the phyto material 419 in more than one surface and preferably contacts the phyto material 419 in at least two perpendicular surfaces, for example, in the form of a bowl where heat from the conduction heating element 107 is applied from a bottom of the phyto material 419 as well as from the sides thereof. Preferably the conduction heating element is manufactured from resistive wire that is attached to an outside of the heating chamber using a capton tape so that it other than contacts the phyto material 419.

Referring to FIG. 1B, when the airflow processing member 106 is coupled at the first end 101c proximate the heating chamber 102 for processing of ambient air that flows through the heating chamber 102, a flow of ambient air that flows through the first fluid pathway 801 is restricted. Preferably the conduction heating element 107 is wrapped about an outside of the heating chamber 102. Further preferably the conduction heating element 107 has a portion of it disposed proximate the second aperture 102b for applying heat to the phyto material 419 from a bottom side of the heating chamber 102, when in use, thus providing heat application to the phyto material 419 from multiple directions to ensure more uniform heat distribution and thus improved vaporization thereof. The airflow processing member 106 is coupled proximate the heating chamber 102, it reduces a flow of ambient air that flows through the heating chamber 102 between the first aperture 102a and the second aperture 102b when the user inhales from the inhalation aperture 103. Reducing the airflow is advantageous as it allows for more restricted inhalation from the inhalation aperture 103 and increases a potency of vaporized phyto material that is mixed with air as the vapor 422. Furthermore, the restriction facilitates a lower temperature of the vapor 422 when inhaled from the inhalation aperture.

Referring to FIG. 1E, an exploded view of the DFV 100 is shown. A temperature sensor 108 is thermally coupled proximate the heating chamber 102 for providing a temperature control signal in dependence upon a temperature of the heating chamber 102. Preferably the heating chamber operates between 100 degrees Celsius and 230 degrees Celsius. A first rechargeable battery 109 is preferably disposed within the housing 101 and coupled with a first control circuit 110 comprising a processor 111, wherein the first control circuit 110 is electrically coupled with the first rechargeable battery 109 and electrically coupled with the conduction heating element 107, the first control circuit 110 is for controlling a flow of electrical current from the first rechargeable battery 109 to the conduction heating element 107 and for reading a voltage of the first rechargeable battery 109 for providing a battery voltage level signal.

For controlling the application of heat to the heating chamber 102, a switch 112 for receiving of tactile input and electrically coupled with the first control circuit 110 for providing a switch control signal to the processor 111. The switch control signal is for affecting the flow of electrical current from the first rechargeable battery 109 to the conduction heating element 107.

At least an indicator LED 113a is electrically coupled with the first control circuit 110, the at least an indicator LED 113a for providing a visual representation of the temperature control signal and the switch control signal and the battery voltage level signal. Referring to FIG. 1F, preferably the at least an indicator LED 113a comprises a plurality of indicator LEDs, 113a, 113b, 113c and 113d. For example these plurality of indicator LEDs are used to display representation of a current temperature of the heating chamber 102.

Referring to FIG. 1D, a charging port 115 is electrically coupled with the first control circuit 110, the charging port 115 is for receiving of electrical energy and for controllably providing of the received electrical energy to the first rechargeable battery 109. Preferably this charging port 115 is a micro USB charging port and is coupled with a standard USB charger as is know in the art for the provision of electrical energy thereto from an eternal supply.

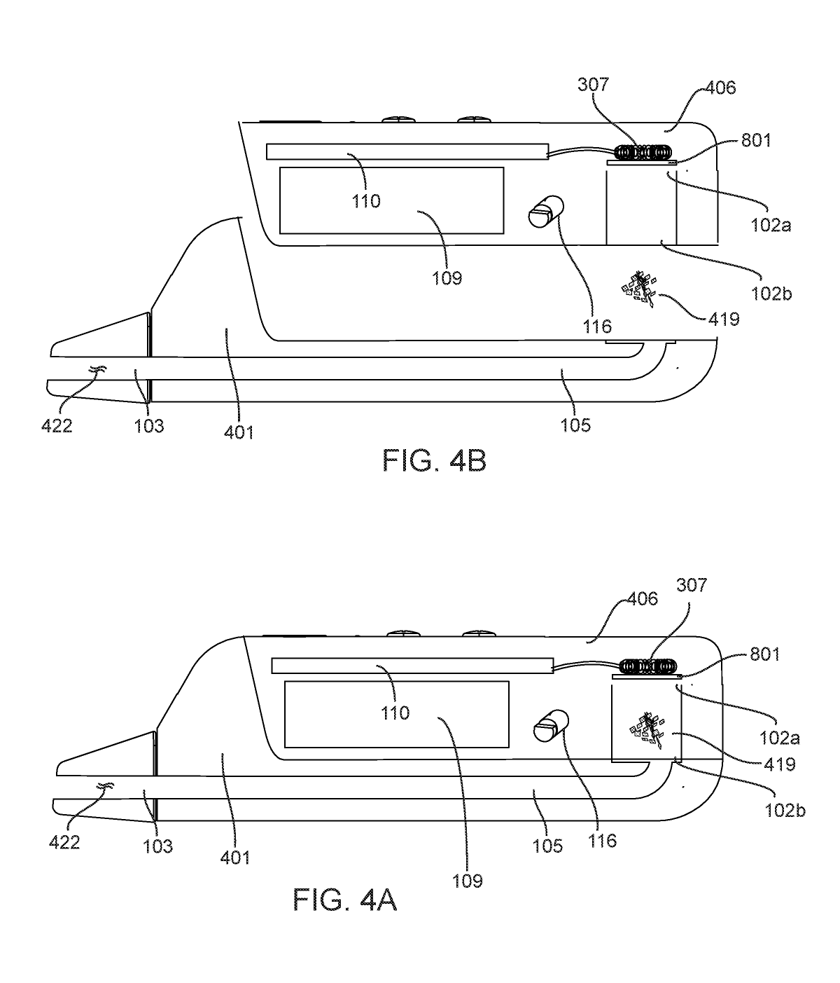

Referring to FIG. 1I, in order to realize an advantages of the invention, a vibration notification system 116 is electrically coupled with the first control circuit 110 and mechanically coupled with one of the housing 101 and the airflow processing member 106 (FIG. 4A) for transmitting of vibration thereto, the vibration notification system 116 for providing the vibration in dependence upon at least one of the switch control signal and the battery voltage level signal and the temperature control signal.

Referring to FIG. 1E, a removable mouthpiece 117 is shown being uncoupled with the second fluid pathway 105 proximate the inhalation aperture 103 and in FIG. 1C the removable mouthpiece 117 is shown being coupled with the second fluid pathway 105 proximate the inhalation aperture 103 and in fluid communication therewith. As is shown in FIG. 1C, for releasably coupling of the airflow processing member 106 to the housing 101. A first magnet 106a is disposed within the airflow processing member 106 and the housing comprises a second magnet 106b, wherein the first and second magnets, 106a and 106b, attract each other when the airflow processing member 106 is coupled with the housing 101 proximate the heating chamber first aperture 102a.

FIGS. 1J and 1I shows the vibration notification system 116 comprising a vibration motor 116a. Preferably the vibration notification system 116 is rigidly coupled with the housing 101 for transmitting of the vibration thereto as is shown in FIG. 1I for being felt and preferably not as much heard by the user.

Referring to FIG. 1I, an insulator material 118 is coupled between the heating chamber 102 and the housing 101 (not shown for clarity), wherein the insulator material 118 is manufactured from a polyamide-imide (PAI) or silicone rubber and the housing 101 is manufactured from a material selected from one of metal and ceramic and plastic and an epoxy mesh. An example of an epoxy mesh would be carbon fiber and in the case of a plastic, preferably a polycarbonate plastic is used where the polycarbonate is preferably food grade. The insulator material 118 serves to protect the housing 101 from the high temperatures of the heating chamber 102. In some cases the heating chamber temperatures will reach 230 degrees Celsius, where some plastics of the housing 101 will soften and deform at this temperature. Having this insulator material 118 formed from silicone rubber or PAI advantageously allows for almost no mechanical deformation of housing 101 when subjected to these temperatures.

Referring to FIG. 1H, preferably in order to maintain heat within the heating chamber 102, a thermal insulating material 119 disposed about an outside surface of the heating chamber 102 for thermally insulating the heating chamber 102 from the housing 101 and for decreasing heat loss from the heating chamber 102 to an outside environment.

Referring to FIGS. 1G and 1H, preferably the second fluid pathway 105 comprises an angled bend along its length between the inhalation aperture 103 and the heating chamber second aperture 102b. Meaning that the first heating chamber aperture 102a is not inline with the inhalation aperture 103, but at an angle thereto. Preferably this angle is approximately ninety degrees. FIG. 1G shows the heating chamber 102 without the thermal insulating material 119 and without the heating element 107.

Referring to FIG. 1J, an audio microphone 120 is mechanically coupled with the second fluid pathway 105 and electrically coupled with the first control circuit 110. The audio microphone 120 is for providing a change in audio signal in dependence upon the flow of air through the second fluid pathway 105 and further through the inhalation aperture 103. When the user inhales through the inhalation aperture 103 a lower air pressure will cause suction and it will result in ambient air flowing into the heating chamber 102 and this will cause an increase in a noise level as picked up by the audio microphone 120, therefore there will be a change in the audio signal in dependence upon air flowing through the second fluid pathway 105 or not. This is useful for determining whether the user is inhaling from the inhalation aperture 103 or not.

Referring to FIG. 1F, the processor 111 disposed as part of the first control circuit 110 comprises a Bluetooth® module 111a, wherein the Bluetooth® module is for wirelessly coupling with a smartphone 88 for having data exchanged therebetween. Such data is derived from at least one of the temperature control signal the switch control signal and the change in audio signal and battery voltage level signal. Optionally the smartphone 88 is for adjusting a temperature of the heating chamber 102 through a smartphone application. Having the smartphone can add another form of a visual cue to the end user for controlling of the DFV.

Referring to FIG. 1K, a tethered connection 121 is shown between an airflow processing member 206 in accordance with a second embodiment of the invention and the housing 101. FIG. 1L shows an electrical connection 122 for electrically coupling of the airflow processing member 206 to the first control circuit 110 and to the housing 101. Optionally the tethered connection is replaced by a hinged connection. Referring to FIG. 1L, a blower system 123 is disposed within the airflow processing member 206, upstream of the first aperture 102a and coupled with the first control circuit 110. The blower system 123 for increasing a flow of ambient air into the heating chamber 102 for propagation through the phyto material 419. In some cases using a blower system 123 is advantageous for those users who do not have sufficient strength in their body to inhale from the inhalation aperture 103, therefore having a system where air is forced into the heating chamber 102 may prove to be advantageous as it will facilitate the inhalation of the vapor 422.

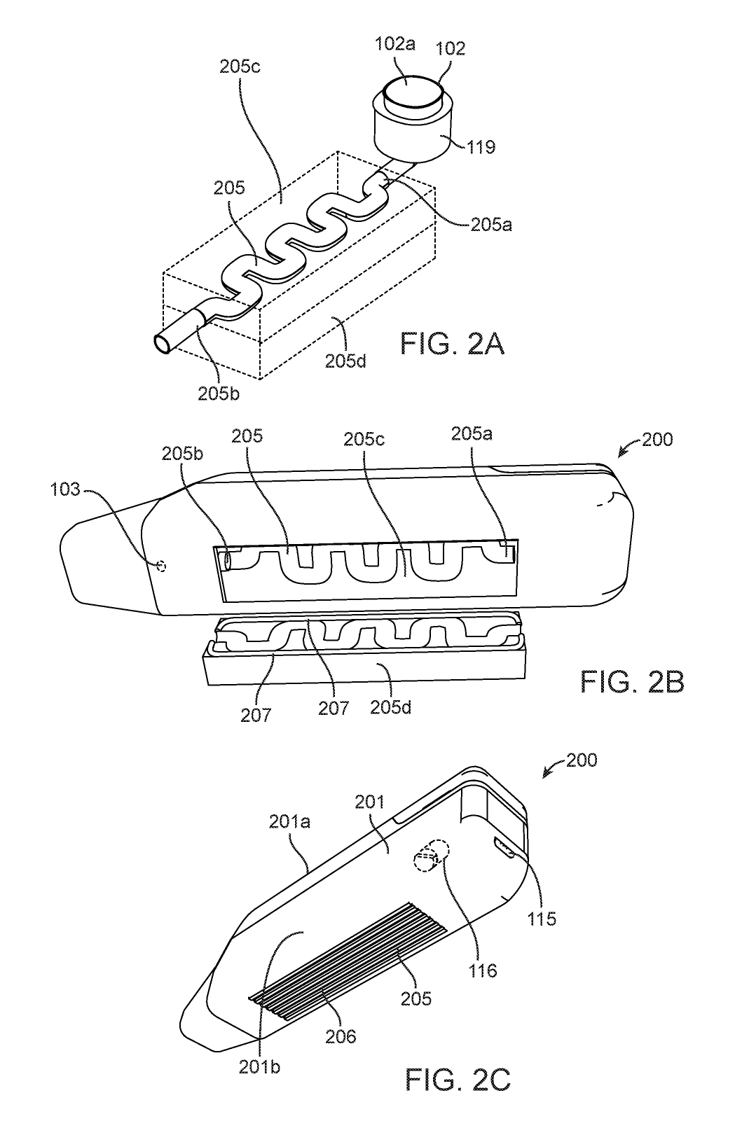

Referring to FIG. 2A, in accordance with a third embodiment of the invention a DFV 200 is shown. In order to reduce a temperature of air flowing through the second fluid pathway 105, preferably a two part second fluid pathway 205 is formed for the DFV 200. As is shown, the two part second fluid pathway 205 comprises a first end 205a and a second end 205b opposite the first end 205a and the two part second fluid pathway 205 comprises a first portion 205c and a second portion 205d, the first and second portions for substantially contacting each other and for being separated from each other. When the first and second portions 205c and 205d are contacting each other the two part second fluid pathway 205 is formed therein and when the first and second portions 205c and 205d are separated from each other allowing for access to an in inside of the two part second fluid pathway 205, as is shown in FIG. 2B. Separating of the two part second fluid pathway 205 allow for cleaning thereof because of easy access to the inside surfaces of both the first portion 205c and the second portion 205d.

Advantageously, the second portion 205d of the two part second fluid pathway 205 comprises a metal material and has a higher thermal conductivity than the first portion 205c. Referring to FIG. 2C, providing fins 206 increases a surface area of the second portion 205d for contacting ambient air and as such increases cooling that is provided to the air flowing through the two part second fluid pathway 205 and function as a heat sink. For facilitating ambient air to substantially not enter the two part second fluid pathway 205, a rubber seal 207 is disposed between the first and second portion, 205c and 205d, for reducing a flow of ambient air from entering into the two part second fluid pathway 205 when other than allowing of ambient air to substantially enter when the first and second portions, 205c and 205d, are substantially contacting each other. Having the two part second fluid pathway 205 facilitates cleaning any residues formed on an inside thereof as a result of vapor propagating therethrough.

FIGS. 3A and 3D illustrate the DFV 400 in accordance with a fourth embodiment of the invention. The DFV 400 is formed from a housing 101 comprising a first end 101c and a second end 101d opposite the first end 101c. A heating chamber 102 for receiving of a phyto material 419 disposed at the first end 101c and an inhalation aperture 103 proximate a second end 101d thereof, the heating chamber 102 comprising a first aperture 102a and a second aperture 102b disposed at an opposite end thereof, a heating chamber fluid pathway 810 formed between the first aperture and the second aperture 102a and 102b. A first fluid pathway 801 for receiving of ambient air is provided and a second fluid pathway 105 is fluidly coupled with the inhalation aperture 103.

An airflow processing member 306 is provided for being releasably coupled at the first end 101c proximate the heating chamber for processing of ambient air that flows through the heating chamber 102. When the airflow processing member 306 is coupled at the first end 101c proximate the heating chamber a continuous fluid pathway 808 is formed from the first fluid pathway 801 through the heating chamber fluid pathway and into the second fluid pathway 105, as shown in FIG. 3D. When the airflow processing member 306 is other than coupled at the first end 101c proximate the heating chamber an other than continuous fluid pathway is formed and allows for loading and unloading of the phyto material 419 into the heating chamber, as shown in FIG. 3A.

Referring to FIGS, 3A, 3B and 3C, a convection heating element 307 is thermally coupled with the first fluid pathway 801 for processing of ambient air by convection heating of air flowing through the first fluid pathway 801 for providing of heated air into the heating chamber 102 for contacting the phyto material 419 to release a vapor from the phyto material for flowing through the second fluid pathway 105 for inhalation through the inhalation aperture 103 when the airflow processing member 306 is coupled at the first end 101c proximate the heating chamber 102. Convection heating, heats up air and causes it to increase in volume and it becomes buoyant and rises. The convection heating element 307 is shown in FIG. 3C disposed within the airflow processing member 306 without a covering screen 852, wherein the covering screen 852 is shown in FIG. 3B as well as an exploded view of the airflow processing member 306 to reveal the convection heating element 307 disposed therein. Preferably a mesh of the covering screen 852 is such that it has holes small enough to not allow the phyto material 419 to substantially contact the convection heating element 307.

FIGS. 4A and 4B illustrate the DFV 500 in accordance with a fifth embodiment of the invention. The DFV 500 is formed from a housing 401 comprising a first end 101c and a second end 101d opposite the first end 101c. A heating chamber 102 for receiving of a phyto material 419 disposed at the first end 101c and an inhalation aperture 103 proximate a second end 101d thereof, the heating chamber 102 comprising a first aperture 102a and a second aperture 102b disposed at an opposite end thereof, a heating chamber fluid pathway 810 formed between the first aperture and the second aperture 102a and 102b. A first fluid pathway 801 for receiving of ambient air is provided and a second fluid pathway 105 is fluidly coupled with the inhalation aperture 103.

An airflow processing member 406 is provided for being releasably coupled at the first end 101c proximate the heating chamber for processing of ambient air that flows through the heating chamber 102. When the airflow processing member 406 is coupled at the first end 101c proximate the heating chamber a continuous fluid pathway 808 is formed from the first fluid pathway 801 through the heating chamber fluid pathway and into the second fluid pathway 105, as shown in FIG. 4A. When the airflow processing member 406 is other than coupled at the first end 101c proximate the heating chamber an other than continuous fluid pathway is formed and allows for loading and unloading of the phyto material 419 into the heating chamber, as shown in FIG. 4B.

A convection heating element 307 is thermally coupled with the first fluid pathway 801 for processing of ambient air by convection heating of air flowing through the first fluid pathway 801 for providing of heated air into the heating chamber 102 for contacting the phyto material 419 to release a vapor from the phyto material for flowing through the second fluid pathway 105 for inhalation through the inhalation aperture 103 when the airflow processing member 406 is coupled at the first end 101c proximate the heating chamber 102. In this embodiment, additionally the first rechargeable battery 109 and the first control circuit 110 and the heating chamber 102 are disposed within the airflow processing member 406.

Advantageously, having the vibration notification system allows facilitates the use of DFV 100 by users who have macular degeneration and other physical limitations that don't allow them to see properly. For example, a single vibration is provided in response to the switch control signal and a double vibration is provided to the battery voltage level signal and a triple vibration is provided in dependence upon the temperature control signal. For example, when a predetermined temperature is reached for the heating chamber then the triple vibration is provided to the user and being indicative of the DFV in accordance with the embodiments of the invention for being ready for use. Similarly upon depressing the switch through tactile input, a vibration notification is provided. Preferably through the smartphone integration through the wireless interface, such as Bluetooth®, various vibration notification patterns are customizable.

Further advantageously, having a restricted flow of ambient air through the first fluid pathway as a result of the airflow processing member provides for a improved vapor density when the vapor is inhaled from the inhalation aperture. As well it advantageously provides for a reduced heat of the vapor when inhaled through the inhalation aperture because the restriction controls a rate of flow of ambient air entering the heating chamber when being processed by the airflow processing member.

The embodiments of the invention advantageously provide for users who suffer from macular degeneration to interact with their DFV for the administering of the aromatherapy in a much simpler manner than that which is available in the prior art. Other conditions that would benefit from vibration notification inclusion in technology also include, but are not limited to: Alzheimer's, Dementias, Muscular Dystrophy, Parkinson's, Depression, Stress, Sleep Deprivation, Head Injuries, Stroke and even certain Medication can also affect vision and short term memory. Haptic feedback, or vibration notification, would again be a gentle reminder to attend to the technology they wish to engage with.

Further advantageously, having the inhalation aperture at an opposite end from the heating chamber allows for increased proximity of the users lips to the heat source and as such can reduce the chances of the user being burned through hot vapor, preferably this distance is at least four centimeters. Additionally advantageous is the two part second fluid pathway that allows for increased cooling of the vapors emitted from the inhalation aperture as well as for easy of cleaning thereof.

Numerous other embodiments may be envisaged without departing from the spirit or the scope of the invention.

What I claim is:

1. A device for vaporizing of phyto material and adapted to fit into a pocket comprising:
a housing comprising a first end and a second end opposite the first end;
a heating chamber for receiving of phyto material disposed upstream and at the first end and an inhalation aperture proximate a second end thereof, the heating chamber comprising a first aperture and comprising a second aperture disposed at an opposite end thereof and downstream from the first aperture, and comprising a heating chamber fluid pathway formed between the first aperture and the second aperture;
a first fluid pathway for receiving of ambient air disposed upstream of the first aperture;
a second fluid pathway fluidly coupled with the inhalation aperture and downstream of the second aperture;
an airflow processing member for restricting a flow of ambient air through the first fluid pathway and being releasably coupled at the first end proximate the heating chamber for processing of ambient air that flows through the heating chamber, wherein when the airflow processing member is coupled at the first end proximate the heating chamber comprising a continuous fluid pathway formed from the first fluid pathway through the heating chamber fluid pathway and into the second fluid pathway and when the airflow processing member is other than coupled at the first end proximate the heating chamber other than comprising the continuous fluid pathway for allowing loading and unloading of the phyto material into the heating chamber;
at least one of a convection heating element and a conduction heating element disposed upstream of the inhalation aperture for heating the phyto material to release a vapor from the phyto material for flowing through the second fluid pathway for inhalation through the inhalation aperture;
a temperature sensor thermally coupled proximate the heating chamber for providing a temperature control signal;
a first rechargeable battery;
a first control circuit comprising a processor, wherein the first control circuit is electrically coupled with the first rechargeable battery and electrically coupled with the at least one of a convection heating element and a conduction heating element, the first control circuit for controlling a flow of electrical current from the first rechargeable battery to the at least a heating element in dependence upon the a temperature control signal and for measuring a voltage level from the first rechargeable battery and for providing a battery voltage level signal;
a switch for receiving of tactile input and electrically coupled with the first control circuit for providing a switch control signal to the first control circuit;
a charging port electrically coupled with the first control circuit, the charging port for receiving of electrical energy and for controllably providing of the received electrical energy to the first rechargeable battery; and,
a vibration notification system electrically coupled with the first control circuit and mechanically coupled with one of the housing and the airflow processing member for transmitting of vibration thereto, the vibration notification system for providing the vibration in dependence upon at least one of the switch control signal and the battery voltage level signal and the temperature control signal, wherein the vibration is for at least partially vibrating of the one of the housing and the airflow processing member.

2. A device for vaporizing of phyto material according to claim 1 wherein the at least one of a convection heating element and a conduction heating element comprises a conduction heating element thermally coupled with the heating chamber which is in contact with the phyto material for conduction heating of the phyto material through transferring of thermal energy thereto, wherein the coupling of the airflow processing member at the first end proximate the heating chamber for processing of ambient air that flows through the heating chamber comprises restricting a flow of ambient air through the first fluid pathway, wherein the conduction heating element operates between 160 degrees Celsius and 230 degrees Celsius.

3. A device for vaporizing of phyto material according to claim 1 wherein the at least one of a convection heating element and a conduction heating element comprises a convection heating element thermally coupled with the first fluid pathway for processing of ambient air by convection heating air flowing through the first fluid pathway for providing of heated air into the heating chamber for contacting the phyto material when the airflow processing member is coupled at the first end proximate the heating chamber. (do I insert a temperature here)

4. A device for vaporizing of phyto material according to claim 1 wherein the airflow processing member comprises one of a tethered coupling to the housing and a hinged coupling to the housing and a twist locking coupling to the housing and a magnetic coupling to the housing.

5. A device for vaporization according to claim 4 wherein the one of tethered connection and hinged connection comprises an electrical connection for electrically coupling of the airflow processing member to the first control circuit.

6. A device for vaporization according to claim 1 comprising an audio microphone mechanically coupled with the second fluid pathway and electrically coupled with the first control circuit, the audio microphone for providing a change in audio signal in dependence upon the flow of air through the second fluid pathway.

7. A device for vaporization according to claim 1 wherein the second fluid pathway comprises two part second fluid pathway comprising a first end and a second end opposite the first end and the two part second fluid pathway comprises a first portion and a second portion disposed between the first end and the second end, the first and second portions for substantially contacting each other and for being separated from each other, wherein when the first and second portions are contacting each other the two part second fluid pathway is formed therein and when the first and second portions are separated from each other allowing for access to an in inside of the two part second fluid pathway for facilitating cleaning thereof.

8. A device for vaporization according to claim 7 wherein the second portion of the two part second fluid pathway comprises a metal material and has a higher thermal conductivity than the first portion.

9. A device for vaporization according to claim 5 comprising a blower system for increasing a flow of ambient air into the heating chamber.

10. A device for vaporizing of phyto material and adapted to fit into a pocket comprising:
a housing comprising a first end and a second end opposite the first end;
a heating chamber for receiving of a phyto material disposed at the first end and an inhalation aperture proximate a second end thereof, the heating chamber comprising a first aperture and a second aperture disposed at an opposite end thereof and downstream from the first aperture, a heating chamber fluid pathway formed between the first aperture and the second aperture;
a first fluid pathway for receiving of ambient air disposed upstream of the first aperture;
a second fluid pathway fluidly coupled with the inhalation aperture and downstream of the second aperture;
an airflow processing member for being releasably coupled at the first end proximate the heating chamber for processing of ambient air that flows through the heating chamber, wherein when the airflow processing member is coupled at the first end proximate the heating chamber a continuous fluid pathway is formed from the first fluid pathway through the heating chamber fluid pathway and into the second fluid pathway and when the airflow processing member is other than coupled at the first end proximate the heating chamber an other than continuous fluid pathway is formed and allows for loading and unloading of the phyto material into the heating chamber;
a conduction heating element disposed upstream of the inhalation aperture and thermally coupled with the heating chamber for conduction heating of the phyto material to release a vapor therefrom for flowing through the second fluid pathway for inhalation through the inhalation aperture, wherein coupling of the airflow processing member at the first end proximate the heating chamber for processing of ambient air that flows through the heating chamber comprises restricting a flow of ambient air through the first fluid pathway into the heating chamber;
a temperature sensor thermally coupled proximate the heating chamber for providing a temperature control signal;
a first rechargeable battery;
a first control circuit comprising a processor, wherein the first control circuit is electrically coupled with the first rechargeable battery and electrically coupled with the heating element, the first control circuit for controlling a flow of electrical current from the first rechargeable battery to the heating element and for measuring a voltage level from the first rechargeable battery and for providing a battery voltage level signal;
a switch for receiving of tactile input and electrically coupled with the first control circuit for providing a switch control signal to the first control circuit;
a charging port electrically coupled with the first control circuit, the charging port for receiving of electrical energy and for controllably providing of the received electrical energy to the first rechargeable battery; and,
a vibration notification system electrically coupled with the first control circuit and mechanically coupled with one of the housing and the airflow processing member for transmitting of vibration thereto, the vibration notification system for providing the vibration in dependence upon at least one of the switch control signal and the battery voltage level signal and the temperature control signal, wherein the vibration is for at least partially vibrating of the one of the housing and the airflow processing member.

11. A device for vaporizing of phyto material according to claim 10 wherein the airflow processing member comprises one of a tethered coupling to the housing and a hinged coupling to the housing and a twist locking coupling to the housing and a magnetic coupling to the housing.

12. A device for vaporization according to claim 11 wherein the one of tethered connection and hinged connection comprises an electrical connection for electrically coupling of the airflow processing member to the first control circuit.

13. A device for vaporization according to claim 10 comprising an audio microphone mechanically coupled with the second fluid pathway and electrically coupled with the first control circuit, the audio microphone for providing a change in audio signal in dependence upon the flow of air through the second fluid pathway.

14. A device for vaporization according to claim 13 wherein the second fluid pathway comprises two part second fluid pathway comprising a first end and a second end opposite the first end and the two part second fluid pathway comprises a first portion and a second portion disposed between the first end and the second end, the first and second portions for substantially contacting each other and for being separated from each other, wherein when the first and second portions are contacting each other the two part second fluid pathway is formed therein and when the first and second portions are separated from each other allowing for access to an in inside of the two part second fluid pathway.

15. A device for vaporization according to claim 14 comprising a linear distance measured between the first end and the second end and the second fluid pathway comprises a path distance as measured along the second fluid pathway between the first end and the second end, wherein the pathe distance is larger than the linear distance.

16. A device for vaporization according to claim 14 wherein the second portion of the two part second fluid pathway comprises a metal material and has a higher thermal conductivity than the first portion.

17. A device for vaporization according to claim 15 comprising a blower system for increasing a flow of ambient air into the heating chamber.

18. A device for vaporizing of phyto material and adapted to fit into a pocket comprising:
a housing comprising a first end and a second end opposite the first end;
a heating chamber for receiving of a phyto material disposed at the first end and an inhalation aperture proximate a second end thereof, the heating chamber comprising a first aperture and a second aperture disposed at an opposite end thereof, a heating chamber fluid pathway formed between the first aperture and the second aperture;
a first fluid pathway for receiving of ambient air disposed upstream of the first aperture;
a second fluid pathway fluidly coupled with the inhalation aperture and downstream of the second aperture;
an airflow processing member for being releasably coupled at the first end proximate the heating chamber for processing of ambient air that flows through the heating chamber, wherein when the airflow processing member is coupled at the first end proximate the heating chamber a continuous fluid pathway is formed from the first fluid pathway through the heating chamber fluid pathway and into the second fluid pathway and when the airflow processing member is other than coupled at the first end proximate the heating chamber an other than continuous fluid pathway is formed and allows for loading and unloading of the phyto material into the heating chamber;
a convection heating element thermally coupled with the first fluid pathway and disposed upstream of the inhalation aperture for processing of ambient air by convection heating air flowing through the first fluid pathway for providing of heated air into the heating chamber for contacting the phyto material to release a vapor from the phyto material for flowing through the second fluid pathway for inhalation through the inhalation aperture when the airflow processing member is coupled at the first end proximate the heating chamber;
a temperature sensor thermally coupled proximate the heating chamber for providing a temperature control signal;
a first rechargeable battery;
a first control circuit comprising a processor, wherein the first control circuit is electrically coupled with the first rechargeable battery and electrically coupled with the heating element, the first control circuit for controlling a flow of electrical current from the first rechargeable battery to the heating element and for measuring a voltage level from the first rechargeable battery and for providing a battery voltage level signal;
a switch for receiving of tactile input and electrically coupled with the first control circuit for providing a switch control signal to the first control circuit;
a charging port electrically coupled with the first control circuit, the charging port for receiving of electrical energy and for controllably providing of the received electrical energy to the first rechargeable battery; and,
a vibration notification system electrically coupled with the first control circuit and mechanically coupled with one of the housing and the airflow processing member for transmitting of vibration thereto, the vibration notification system for providing the vibration in dependence upon at least one of the switch control signal and the battery voltage level signal and the temperature control signal, wherein the vibration is for at least partially vibrating of the one of the housing and the airflow processing member.

19. A device for vaporizing of phyto material according to claim 18 wherein the airflow processing member comprises one of a tethered coupling to the housing and a hinged coupling to the housing and a twist locking coupling to the housing and a magnetic coupling to the housing.

20. A device for vaporization according to claim 18 wherein the second fluid pathway comprises two part second fluid pathway comprising a first end and a second end opposite the first end and the two part second fluid pathway comprises a first portion and a second portion disposed between the first end and the second end, the first and second portions for substantially contacting each other and for being separated from each other, wherein when the first and second portions are contacting each other the two part second fluid pathway is formed therein and when the first and second portions are separated from each other allowing for access to an in inside of the two part second fluid pathway.
